(19) United States
(12) Patent Application Publication
(10) Pub. No.: US 2014/0170236 A1
(43) Pub. Date: Jun. 19, 2014

(54) USE OF PHOSPHORIC ACID

(71) Applicants: Priscilla Hayes Petty, Nashville, TN (US); Gene Paul Petty, Nashville, TN (US); Damon Hayes Petty, Nashville, TN (US)

(72) Inventors: Priscilla Hayes Petty, Nashville, TN (US); Gene Paul Petty, Nashville, TN (US); Damon Hayes Petty, Nashville, TN (US)

(21) Appl. No.: 14/002,667

(22) PCT Filed: Nov. 4, 2011
(86) PCT No.: PCT/US11/59436

§ 371 Date: Jan. 14, 2014

Related U.S. Application Data
(60) Provisional application No. 61/410,805, filed on Nov. 5, 2010.

(51) Int. Cl. A61K 33/42; A61K 31/525; A61K 31/047

(52) U.S. Cl. CPC A61K 33/42; A61K 31/047; A61K 31/525
USPC 424/605; 423/317

(57) ABSTRACT

This disclosure relates to the use of phosphoric acid. In particular, this disclosure relates to the use of phosphoric acid for treating cardiovascular disorders such as atherosclerosis. This disclosure further relates to compositions, kits, methods, and the like.

### FIELD

This disclosure relates to the use of phosphoric acid. In particular, this disclosure relates to the use of phosphoric acid for treating cardiovascular disorders such as atherosclerosis. This disclosure further relates to compositions, kits, methods, and the like.

### BACKGROUND

Cardiovascular diseases are a group of disorders of the heart and blood vessels that includes coronary artery disease (disease of the blood vessels supplying the heart muscle), cerebrovascular disease (disease of the blood vessels supplying the brain), deep vein thrombosis and pulmonary embolism (blood clots in the veins), and peripheral vascular disease (disease of blood vessels supplying the arms and legs). Heart attacks and strokes are usually acute events and are mainly caused by a blockage that prevents blood from flowing to the heart or brain. The most common reason for this is a build-up of fatty deposits on the inner walls of the blood vessels that supply the heart or brain and the development of atherosclerotic plaques that can cause platelet aggregation in the narrowed artery and block blood flow to the terminal organ (heart, brain, other organ or extremity). Strokes can also be caused by bleeding from a blood vessel in the brain or from blood clots.

Cardiovascular diseases are the number one cause of death globally, claiming 17.1 million lives a year. Deaths from cardiovascular diseases are projected to reach 23.6 million by the year 2030. Tobacco use, an unhealthy diet, and physical inactivity increase the risk of heart attacks and strokes. Low-and middle-income countries are disproportionally affected, making up 82% of cardiovascular disease deaths. Increased exposure to risk factors, less access to health care services, and a lack of effective preventative programs seem to be the main reasons for the high incidence of cardiovascular disease in low to middle income countries. In addition to the obvious societal impact, cardiovascular diseases place a heavy burden on the economies of such countries. For example, it is estimated that China will lose approximately $558 billion in foregone national income due to the combination of heart disease, stroke, and diabetes.

While the risk of heart disease and stroke can be reduced through lifestyle and dietary choices there are also several medicaments designed to treat cardiovascular diseases. For example, statins can be used to lower cholesterol, low-dose aspirin can slow blood clotting by reducing clumping of platelets, and there are various medications for controlling blood pressure (e.g. beta-blockers, ACE inhibitors). In addition to drug therapy there are several surgical options such as, for example, coronary artery bypass, balloon angioplasty, stents, valve repair and replacement, heart transplantation, and artificial heart operations. Despite the numerous options available many patients fail to maintain their drug regimens due to the significant side-effects and consequent loss of quality of life.

There exists a need for further safe and effective treatment options for cardiovascular diseases. For example, although widely used and capable of reducing the growth of non-calcified plaques, statins do not significantly affect the growth rate of mixed or calcified plaques (Hoffmann H. et al., Eur Radiol (2010 Jul. 18) “Influence of statin treatment on coronary atherosclerosis visualised using multidetector computed tomography”). There exists a need for treatment options with reduced impact on the subject's quality of life. There exists a need for treatment options for cardiovascular disease which may be easily distributed to low- and middle-income countries.

### SUMMARY

The present disclosure provides the use of phosphoric acid for the treatment of cardiovascular disorders such as atherosclerosis.

The present disclosure further provides compositions, kits, and methods relating to the use of phosphoric acid for treating cardiovascular disorders.

The present disclosure further provides the use of phosphoric acid for the manufacture of a medicament for the treatment of a cardiovascular disorder.

The present disclosure further provides the use of phosphoric acid fior the treatment of calcified, soft, or mixed atherosclerotic plaque.

As used herein, the term “cardiovascular condition” refers to any disease, ailment or malady that affects the cardiovascular system and is caused by the deposit of fatty material, such as cholesterol, or the formation of atherosclerotic plaques (including calcified plaque) in blood vessels. Examples of cardiovascular conditions include, but are not limited to, coronary artery disease, cerebrovascular disease, deep vein thrombosis, pulmonary embolism, and peripheral arterial disease.

As used herein, the term “treatment” means any manner in which a cardiovascular condition in a subject is eliminated, ameliorated, or otherwise beneficially altered. “Treatment” as used herein is intended to encompasses prophylactic use of phosphoric acid to reduce or eliminate the risk of a cardiovascular disease condition developing or progressing.

As used herein, the term “subject” is not limited to a specific species or sample type. For example, the term “subject” may refer to a patient, and frequently a human patient. However, this term is not limited to humans and thus encompasses a variety of mammalian species.

As used herein, “a” or “an” means “at least one” or “one or more”.

This summary does not necessarily describe all features of the invention. Other aspects, features and advantages of the invention will be apparent to those of ordinary skill the art upon review of the following description of specific embodiments of the invention.

### DETAILED DESCRIPTION

The present disclosure provides the use of phosphoric acid for treatment of cardiovascular disease disorders. For example, phosphoric acid may be used to treat sclerosis of the cardiovascular systems such as arteriosclerosis, arteriolosclerosis, or atherosclerosis.

It has been found that under certain circumstances phosphoric acid can reduce or eliminate the soft plaque that accumulates in blood vessels. It has also been found that under certain circumstances phosphoric acid can stop, slow, or even reverse, the build-up of calcified plaque deposits in blood vessels. Calcified plaque is responsible for the hardening and reduced diameter of diseased arteries. While not wishing to be bound by theory, it is believed that (1) the phosphoric acid reduces the amount of soft arterial plaque that is the precursor of calcified plaque, thus preventing or reducing the formation of additional calcified plaque; and/or (2) the phosphoric acid binds the calcium that would normally bind with cholesterol to form calcified plaque, thereby preventing the formation of new calcified plaque; and/or (3) over time the calcium in the hard plaque may be bound by the phosphorus, so that plaque may be at least partially eliminated from the body.

A coronary computed tomography (CT) calcium scan is a test to assess the build up of plaque (calcification) in the coronary arteries. Because calcium is a component of hard plaque and is a marker of coronary artery disease (CAD) such a scan can be used as a diagnostic tool for CAD. The amount of calcium detected on a CT scan, expressed as a calcium score, reflects the degree and extent of calcium deposits on the walls of coronary arteries. Once calcified plaque begins to accumulate on arterial walls the amount deposited increases logarithmically at a rate of 15% to 30% a year. It has been found that ingestion of phosphoric acid can prevent the usual continual increase in calcium score over time or reduce the calcium score in a subject suffering from CAD.

The calcium score is an indicator of potential heart risk. The American College of Cardiology in March 2009 stated in a release that “Coronary calcium scoring can help predict who is likely to have a heart attack or die of coronary artery disease, according to research presented today (Mar. 29, 2009) at the American College of Cardiology's 58^{th} Annual Scientific Session.” The study “found that the coronary artery calcium score was better predictor of heart disease than classic risk factors at predicting risk over five years.” Calcium scores can range from 0 into the hundreds and even into the thousands. A higher score is indicative of an increased risk and cardiologists express concern of the possibility of a heart incident at various levels but especially at 400 or above. Some treatments such as the product Crestor®, claim to slow the rate of deposition of plaque. Unfortunately Crestor® has a risk of unwanted side effects and at the present time there is no other known treatment which stops or reverses the deposition of calcified plaque in the arteries.

Phosphoric acid (e.g. H₃PO₄) is non-toxic and highly water soluble. The substance has many uses including as a food and beverage additive, as an orthodontic etching solution, in teeth whitening products, in cleaning solutions for removal of mineral deposits, in microfabrication to etch silicon nitride, as an acidifier in cosmetic compositions. Phosphoric acid is also used as an excipient in certain pharmaceutical preparations.

Phosphoric acid is widely available from a variety of sources. Preferred for use herein is orthophosphoric acid. For example, Phosphorex™, by DNA Laboratories, is a liquid preparation containing phosphoric acid, inositol, riboflavin, and water; Phosphoric Acid, by Progressive Laboratories, and Phosfood®, by Standard Process, also contain orthophosphoric acid, inositol, riboflavin, and water.

Any suitable dosage may be used herein. Dosages for any one patient depends upon many factors, including the subject's size, body surface area, age, the particular composition to be administered, sex, time and route of administration, general health, and other drugs being administered concurrently. Preferably the subject takes a daily dose of phosphoric acid. Preferably the daily dose is at least about 1 mg or greater of phosphorus such as, for example, about 5 mg or greater, about 25 mg or greater, about 50 mg or greater, about 75 mg or greater, about 100 mg or greater, about 120 mg or greater, about 130 mg or greater, about 140 mg or greater, about 150 mg or greater, about 160 mg or greater, about 170 mg or greater, about 180 mg or greater, about 190 mg or greater, about 200 mg or greater, about 210 mg or greater, about 220 mg or greater, about 230 mg or greater, about 240 mg or greater, about 250 mg or greater, about 260 mg or greater, about 270 mg or greater, of phosphorus.

The phosphoric acid may be delivered in aqueous form. For example, the aqueous phosphoric acid composition may comprise at least about 1%, at least about 5%, at least about 10%, at least about 15%, at least about 20%, at least about 50%, by weight, of phosphoric acid.

The phosphoric acid may be delivered in an anhydrous form. For example, anhydrous phosphoric acid powder may be compounded in capsules, tablets or the like and taken with water.

The phosphoric acid may be delivered via any suitable route of administration. For example, the phosphoric acid composition may be ingested orally. Or it may be possible to deliver phosphorus in another form such as, for example, intravenously.

The present disclosure is also directed to compositions comprising phosphoric acid. Any suitable composition may be used herein. The compositions may be in any suitable form such as, for example, a powder, capsule, tablet, wafer, paste, gel, liquid, or suspension. Preferably the phosphoric acid is in the form of a liquid, capsule, or tablet.

The present composition must comprise phosphoric acid but may, in addition, comprise a variety of optional ingredients. For example, the composition may comprise a pharmaceutically acceptable carrier. As used herein “pharmaceutically acceptable carrier” means a composition suitable for pharmaceutical administration. Examples of pharmaceutically acceptable carriers include saline, solvents, dispersion media, coatings, antibacterial and antifungal agents, isotonic and absorption delaying agents, and the like. Pharmaceutically acceptable carriers will typically comprise one or more excipients. Any suitable food or pharmaceutical excipient may be used herein. Examples of pharmaceutical excipients may be found in The Handbook of Pharmaceutical Excipients, R. C. Rowe et al., 6^{th} Edition, Pharmaceutical Press.

The present composition may comprise water. Preferably, the composition comprises water in an amount of at least about 10% or greater, about 20% or greater, about 30% or greater, about 40% or greater, about 50% or greater, about 60% or greater, about 70% or greater, about 80% or greater, about 90% or greater, about 99%, by weight of total composition.

The present composition may comprise one or more types of vitamin. For example, vitamin B2 (riboflavin). The vitamin may be present in any suitable amount such as, for example, about 50% or less, about 20% or less, about 10% or less, about 5% or less, about 1% or less, about 0.8% or less, about 0.5% or less, about 0.25% or less, by weight of total composition.

The present composition may comprise inositol. The inositol may be present in any suitable amount such as, for example, about 50% or less, about 20% or less, about 10% or less, about 5% or less, about 1% or less, about 0.8% or less, about 0.5% or less, about 0.25% or less, by weight of total composition.

If desired, additional active compounds may also be incorporated into the compositions or may be delivered separately but contemporaneously. For example, niacin, niacinamide, and other forms of niacin; folate, choline, B12 other B vitamins; aspirin, or the inclusion of other known agents that positively affect cardiovascular health, such as HMGCCoA-reductase inhibitors, or blood pressure control agents.

The present composition may comprise a substance having antioxidant properties such as, for example, krill oil, fish oil, or combinations thereof. Or such substances may be delivered separately but contemporaneously.

As used herein, the term “contemporaneously” means within the same time period. For example, during the same day, within an hour, or simultaneously.

The present compositions may be presented in the form of a “functional food.” For example, the present composition can be a cookie, chocolate, candy, bread, ice cream, yogurt, sport drinks, juice, meal replacement drinks, soft drinks and other types of drinks, and the like. These product presentations can be useful to help subjects who are reluctant to take a capsule or tablet to ingest the product. Preferably, each functional food comprises a dosage of at least about 1 mg or greater of phosphorus such as, for example, about 5 mg or greater, about 2.5 mg or greater, about 50 mg or greater, about 75 mg or greater, about 100 mg or greater, about 120 mg or greater, about 130 mg or greater, about 140 mg or greater, about 150 mg or greater, about 160 mg or greater, about 170 mg or greater, about 180 mg or greater, about 190 mg or greater, about 200 mg or greater, about 210 mg or greater, about 220 mg or greater, about 230 mg or greater, about 240 mg or greater, about 250 mg or greater, about 260 mg or greater, about 270 mg or greater, of phosphorus.

The present invention also relates to kits comprising the composition. The present kit preferably comprises the composition as described herein and instructions for administering said composition to a subject having a cardiovascular disease. Preferably the instructions recommend ingestion on a regular, preferably daily basis. The present kits may comprise other compositions. If present, the other compositions preferably comprise aspirin.

It is contemplated that any part of any aspect or embodiment discussed in this specification can be implemented or combined with any other embodiment, aspect, method, composition or aspect of the invention.

Unless defined otherwise, all technical and scientific terms used herein have the same meaning as is commonly understood by one of ordinary skill in the art to which this invention belongs. Unless otherwise specified, all patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference. Citation of references herein is not to be construed nor considered as an admission that such references are prior art to the present invention.

Use of examples in the specification, including examples of terms, is for illustrative purposes only and is not intended to limit the scope and meaning of the embodiments of the invention herein. Numeric ranges are inclusive of the numbers defining the range. In the specification, the word “comprising” is used as an open-ended term, substantially equivalent to the phrase “including, but not limited to,” and the word “comprises” has a corresponding meaning.

The invention includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention order to achieve the same result in substantially the same way.

The present invention will be further illustrated in the following examples. However it is to be understood that these examples are for illustrative purposes only, and should not be used to limit e scope of the present invention in any manner.

### EXAMPLES

### Example 1

A 68 year old man with no previous history of cardiovascular disease suffered from a coronary incident. Upon investigation it was found that the subject's left anterior descending artery was 98% blocked and a cardiac interventionist placed a bare metal stent in the artery.

The subject was prescribed various medications during the post-operative period including a combination of platelet inhibitors, cholesterol-reducing stains, and blood pressure medications such as Plavix®, Aspirin, Lipitor®, verapamil, and Lescol®.

The subject suffered from extreme physical fatigue, mental fogginess, dizziness, muscle pain and less interest in physical or intellectual pursuits. The subject maintained the drug regimen for several months but suffered from significant loss of quality of life and eventually stopped taking the medications except aspirin.

Approximately eleven months after the placement of the stent the subject suffered from chest pains and a further investigation revealed that the stent was 60% occluded. The angiogram taken at this time showed extensive calcified plaque. The cardiac interventionist prescribed the same kind of drug therapy as above. A stress test was conducted at this time to provide a baseline for comparing with possible future stress tests. Results of this stress test were positive, confirming a cardiac problem. Despite his best efforts the subject could not maintain the regimen and only continued with low dose aspirin.

The subject then took a 2.5 mL daily dose of a 23.8% (mg/L) solution of phosphoric acid composition comprising 183 mg of phosphorus for a period of twelve months. This represented 26% of the RDA of phosphorous (700 mg) for an adult. A second stress echo test was conducted at this time at the same hospital and the results were negative, indicating that the coronary condition had improved. The subject's calcium score after one year of this regimen was 29 and the probability of a heart attack in the next 10 years was estimated to be 10%. The CT calcium scan report stated that his arteries were those of a 54-year-old male rather than a 69-year-old male. Although there was no base CT calcium score it would be rare for a male of his cardiac history and age to have a calcium score this low. Two years after the angiogram that showed the extensive calcified plaque, a CT Angiogram was performed that showed no calcified plaque except in the area of the stent.

The subject suffered from no side-effects related to the phosphoric acid composition and a bone scan taken at this time showed normal bone density.

### Example 2

A 69 year old woman with no previous history of cardiovascular disease had a CT calcium scan and was assessed a calcium score of 181. The calcium scan report said that this score indicated that her arteries were fifteen years older than her age because of calcium plaque deposition and that she was at 44% risk of a heart attack within ten years. The risk assessment assumed that the calcified plaque would increase at the usual anticipated rate of approximately 15% to 30% a year. She took a daily dose of 2.5 ml of a 23.8% (mg/L) phosphoric acid in water, (183 mg of phosphorus) for a period of six months. A second CT scan at six months revealed a calcium score which remained steady at 181. The subject then increased the daily dose of phosphoric acid to 3.75 ml (274 mg of phosphorus) and suffered no side effects at the higher level. A third CT calcium scan was performed approximately three months after increasing the dose. This was eight months and twenty days after the initial dosing with phosphoric acid. The calcium score was 179. Since arterial plaque deposits at a logarithmic rate, the subject's predicted calcium score at this time would have been 197 (at an average rate of 19% increase/year). The reduction in calcium score from 181 to 179 is not statistically significant but the cessation of additional plaque deposition is noteworthy because the calcium score did not rise as would be expected. The importance of the cessation could have enormous implications for coronary artery disease treatment.

At the same time the most recent CT calcium scan was done, a Computed Tomography Angiography (CTA) scan was also done with contrast dye to determine the amount of soft plaque in the arteries. The resulting soft plaque score was 0 (zero), indicating that no soft plaque was present in the arteries. This indicates that the phosphoric acid solution, at the dosage taken, could have the effect of reducing or removing soft plaque and the calcium associated with it, thereby reducing the risk of the development of additional hard calcified arterial plaque. Although no CTA scan was taken before beginning to take the phosphoric acid solution, soft plaque is assumed to have previously been present since soft plaque is a precursor to hard calcified plaque. Thus, the significant amount of calcified plaque that existed before beginning the phosphoric acid solution treatment, as indicated by the initial CT Calcium score of 181, would not have been present had there not been soft plaque to be converted.

### Example 3

A 63 year old male former smoker with a cardiac history of balloon angioplasty surgery 22 years earlier had a CT Calcium Scan and was assessed with a calcium score of 193 and a 54% probability of a heart attack in the next 10 years. The subject takes Famotadine for acid reflux and continues to smoke “one or two cigars a week.” The subject began taking a daily dose of 3.75 ml of a 23.8% (mg/L) phosphoric acid solution. A second CT Calcium Scan was taken approximately six months after beginning to take the phosphoric acid and the score was 122, a 37% reduction. The expected score after 6 months (at a 19% increase per year) would have been 262.

### Example 4

A 54 year old non-smoking male (except for “about one cigar a month”) with a history of hypertension, elevated cholesterol, heartburn and heart palpitations had a CT Calcium Scan and was assessed with a calcium score of 85 and a 54% probability of a heart attack in the next 10 years. He currently takes lipid lowering medication. He began taking 3.75 ml of a 23.8% (mg/L) phosphoric acid solution shortly after his CT Calcium Scan. A second CT Calcium Scan was taken eight months after beginning to take phosphoric acid and the score was 83, or 2 points lower than the initial score. The expected score after 6 months (at a typical 19% increase per year) would have been 115.

### Example 5

A 55 year old male, heavy smoker, with a history of hypertension and elevated cholesterol had an initial CT Calcium Score of 117. His 10-year risk of a heart attack was estimated to be 99%. His medications includes: Benicar®, Crestor®, Niaspan®, and Lovaza®. Two and a half months after having the first Calcium Score assessment he began taking a daily dose of 3.75 ml of a 23.8% (mg/L) phosphoric acid solution. A second CT Calcium Scan eight months after he began taking phosphoric acid showed a score of 130, an increase of 11%, or an annual rate of increase of 13.4%. The predicted score after 8 months (at a typical 19% increase per year) would have been 197. Note, cadaver studies consistently indicate that smokers are more prone to advanced artery disease. It is not clear what effect the delay in starting therapy and smoking habits had to this subjects calcium score. However, the fact that the subject's score increased at a lower rate than predicted is indicative of a beneficial effect of the phosphoric acid treatment.

### SUMMARY

Of the four non-smoking subjects, none experienced an increase in Calcium Score and one of the four experienced a dramatic decrease after only six months of taking the phosphoric acid. The subject who smoked experienced an increase in calcified plaque although this was lower than the predicted rate of increase. For perspective, the table below shows a comparison of subjects who took phosphoric acid and a study of 913 subjects who did not take phosphoric acid, and 56% of whom were taking traditional cardiac medicines.

| | Cardiologists' | Phosphoric |
|---|---|---|
| | Data | Acid Users |
| Non-Smokers | | |
| (Sample size) | (586) | (4)^{Note 1} |
| % Whose Ca Score Increased | 86% | 0% |
| Smokers | | |
| (Sample size) | (327) | (1) |
| % Whose Ca Score Increased | 92% | 100% |
| All | | |
| (Sample size) | (903) | (5) |
| % Whose Ca Score Increased | 88% | 20% |

^{Note 1}
Three of the four subjects had both pre study and follow up Calcium Scores. The 4^{th} subject (Example #1) is included based on a follow up CT Angiogram that shows no calcified plaque except in the area of a stent.

1.-11. (canceled)

12. A kit comprising a composition comprising phosphoric acid and instructions for ingesting said composition by a subject having, or desiring to prevent, a cardiovascular disease, wherein said instructions indicate a daily dose of at least about 50 mg of phosphorus in the form of phosphoric acid.

13. The kit of claim 12:
wherein said composition comprises phosphoric acid, inositol, and riboflavin.

14. A method of preventing, stopping, slowing, or reducing cardiovascular plaque deposition in a subject in need thereof, the method comprising administering to the subject an efficacious dose of anhydrous phosphoric acid.

15. A method of preventing, stopping, slowing, or reducing cardiovascular plaque deposition in a subject in need thereof, the method comprising administering to the subject at least about 50 mg per day of phosphorus in the of phosphoric acid.

16. The method of claim 15, the method comprising administering to the subject at least about 100 mg per day of phosphorus in the form of phosphoric acid.

17. The method of claim 15, the method comprising administering to the subject at least about 150 mg per day of phosphorus in the form of phosphoric acid.

18. The method of claim 15, the method comprising administering to the subject a composition comprising phosphoric acid and inositol.

19. The method of claim 15, the method comprising administering to the subject a composition comprising phosphoric acid, inositol, and riboflavin.

20. The method of claim 14 wherein the cardiovascular plaque is arterial soft plaque.

21. The method of claim 15 wherein the cardiovascular plaque is arterial soft plaque.
